(19) United States
(12) Patent Application Publication
(10) Pub. No.: US 2013/0129652 A1
(43) Pub. Date: May 23, 2013

(54) COLORED AQUEOUS COSMETIC COMPOSITIONS

(71) Applicants: Xavier Blin, Paris (FR); Laurence Guerchet, Verrieres-le-Buisson (FR); Anne Marie Lezoray, Villeconin (FR)

(72) Inventors: Xavier Blin, Paris (FR); Laurence Guerchet, Verrieres-le-Buisson (FR); Anne Marie Lezoray, Villeconin (FR)

(73) Assignee: L'OREAL, Paris (FR)

(21) Appl. No.: 13/702,461

(22) PCT Filed: Jun. 8, 2011
(86) PCT No.: PCT/IB2011/052496

§ 371 Date: Jan. 28, 2013

Related U.S. Application Data
(60) Provisional application No. 61/379,053, filed on Sep. 1, 2010.
(60) Provisional application No. 61/379,060, filed on Sep. 1, 2010.

(30) Foreign Application Priority Data
Jun. 8, 2010 (FR) 1054498
Jun. 8, 2010 (FR) 1054499

(51) Int. Cl. A61K 8/04; A61Q 1/02

(52) U.S. Cl. CPC A61K 8/04; A61Q 1/02
USPC 424/63; 424/70.6

(57) ABSTRACT

The present invention relates to a cosmetic care and/or makeup composition comprising at least 65% by weight relative to its total weight of an aqueous phase, at least 2% by weight relative to its total weight of pigments in the form of pigmentary particles that are dispersed and/or dispersible in aqueous medium, at least one water-soluble or water-dispersible gelling agent in a content ranging from 0.05% to 40% by weight relative to the total weight of the composition, and at least one polyol containing from 3 to 12 carbon atoms, said composition having a viscosity at least equal to 600 Pa·s at a shear of 0.010 s—and less than 0.8 Pa·s at a shear of 1000 s—, and said pigmentary particles being first formed from an intimate mixture of said pigments and from at least one polyol.

The present invention is directed toward proposing formulations that are more particularly intended for making up facial skin, which combine the makeup properties of a foundation and the sensory and/or efficacy properties of a care product of aqueous architecture.

In general, the majority of women, and nowadays even men, particularly appreciate care products of aqueous architecture with regard to the sensory properties experienced on application of this type of architecture. The reason for this is that their texture is very light and they prove to be very easy to apply given their slippery nature, or even are also capable of affording a sensation of freshness.

Unfortunately, reproducing this architecture with a makeup formula, which generally conveys a certain amount of particulate material, including pigments intended to give the composition a color effect, is not entirely satisfactory. An aqueous architecture, which is advantageous with regard to the fresh and slippery properties it imparts, does not ensure uniform distribution of the particulate materials within the composition. To overcome this defect, these makeup compositions generally also contain a significant amount of fatty substances. Thus, makeup formulations such as foundations are water-in-oil or water-in-silicone emulsions, stabilized with surfactants, the water content of which is conventionally from 20% to 30%, up to 40%, rarely greater than 50% and never greater than 60%.

These architectures of emulsion type enable the particles they contain to be uniformly stabilized. Moreover, with regard to their thicker structure, they allow the user to take up more makeup product in a single action, which is an option that is more difficult to achieve with aqueous formulations that are much more liquid

However, these two advantages in this type of formulation are obtained at the expense of the fresh and/or slippery aspect, which is conditioned by the amount of water present in the composition. For obvious reasons, it would be advantageous for users to combine in a novel formulation the respective qualities of aqueous formulations, namely freshness and softness, and of formulations of foundation type, in terms of dispersion of particulate materials and uptake. Unexpectedly, the inventors have found that such an objective may be achieved provided that specific compounds are combined in an aqueous architecture.

In particular, the present invention is directed toward protecting a makeup cosmetic product especially of foundation type that is applied in a single action and that combines with its makeup performance the true sensory properties of a care product with regard to its significant content of aqueous medium, while at the same time ensuring good stability over time of the dispersed pigments.

More particularly, the present invention relates to a cosmetic care and/or makeup composition comprising at least 65% by weight of an aqueous phase, at least 2% by weight relative to its total weight of pigments in the form of pigmentary particles that are dispersed or dispersible in aqueous medium, and at least one water-soluble or water-dispersible gelling agent in a content ranging from 0.05% to 40% by weight relative to the total weight of the composition.

Thus, according to one of its aspects, the present invention relates to a cosmetic care and/or makeup composition comprising at least 65% by weight of an aqueous phase, at least 2% by weight relative to its total weight of pigments in the form of pigmentary particles that are dispersed or dispersible in aqueous medium, at least one water-soluble or water-dispersible gelling agent in a content ranging from 0.05% to 40% by weight relative to the total weight of the composition, and at least one polyol containing from 3 to 12 carbon atoms, said composition having a viscosity at least equal to 600 Pa·s at a shear of 0.010 s⁻¹ and less than 0.8 Pa·s at a shear of 1000 s⁻¹, and said pigmentary particles being first formed from an intimate mixture of said pigments and from at least one polyol.

According to another of its aspects, the present invention relates to a cosmetic care and/or makeup composition comprising at least 65% by weight of an aqueous phase, at least 2% by weight relative to its total weight of pigments in the form of pigmentary particles that are dispersed or dispersible in aqueous medium, at least one water-soluble or water-dispersible gelling agent in a content ranging from 0.05% to 40% by weight relative to the total weight of the composition, and at least one polyol containing from 3 to 12 carbon atoms, said composition having a viscosity at least equal to 600 Pa·s at a shear of 0.010 s⁻¹ and less than 0.8 Pa·s at a shear of 1000 s⁻¹, said gelling agent being chosen from modified or unmodified carboxyvinyl polymers, polyacrylates and polymethacrylates, optionally crosslinked and/or neutralized 2-acrylamido-2-methylpropanesulfonic acid polymers and copolymers, ethoxylated (25 EO) AMPS/stearyl methacrylate copolymers crosslinked with trimethylolpropane triacrylate (TMPTA), AMPS/acrylamide copolymers, polyacrylamides, associative polyurethanes, polysaccharides such as xanthan gum, xanthan derivatives, carrageenans, gellans, alginates, celluloses such as microcrystalline cellulose and cellulose polymers such as hydroxyethylcellulose, hydroxymethylcellulose, hydroxypropylcellulose, methylcellulose, ethylhydroxyethylcellulose and carboxymethylcellulose, and mixtures thereof.

Advantageously, the particles of such a composition are first formed from an intimate mixture of said pigments and from at least one polyol.

The presence of the gelling agent is advantageous in several respects.

First, by virtue of its combination with a significant amount of water, it affords a composition that may be likened to a texture said to have a “quick-break” effect.

Thus, when the composition as defined above is applied to the skin, its structure breaks, giving a pleasant fresh effect known as the “quick break” due to the release of the water of the formulation.

Furthermore, it contributes efficiently toward the homogeneous dispersion of the pigments within the composition.

Indeed, for a majority of pigment types, a problem of aggregation of the particles of which they are constituted arises. Now, such aggregates, which may be likened to large particles, have no dispersible nature and are thus subject to rapid sedimentation when they are used in an aqueous phase. This results in totally inhomogeneous distribution of said pigments within said aqueous medium.

The gelling agent present in the compositions of the invention ensures the stability of the dispersed state of the pigments by especially preventing their sedimentation.

Thus, in a composition according to the invention, the pigments are present in an individualized particulate form, i.e. not aggregated, and dispersed or dispersible in aqueous medium. This optimization of dispersion, ensured especially by the presence of the gelling agent, makes it possible to ensure homogeneity over time of the color effect that these pigments need to afford.

In addition, the inventors have found that it is possible to reinforce this dispersion in particulate form, provided that the pigments are used in a form combined with at least one polyol.

Thus, according to one embodiment variant, the pigmentary particles are formed from said pigments and from at least one polyol in a polyol(s)/pigments ratio ranging from 0.025 to 7.5, in particular ranging from 0.05 to 7 and more particularly from 0.1 to 6.

Manifestly, the polyol also has the effect of efficiently contributing toward the dispersion and/or dispersibility of the pigments under consideration according to the invention in the form of fine particles not subject to aggregation, within an aqueous composition as considered according to the invention.

Specifically, the pigments under consideration according to the invention in a form combined with the polyol, which may also be termed as being wetted with the polyol, prove, in the absence thereof, to be dispersed in the form of small particles in a homogeneous and more stabilized manner in an aqueous medium.

More particularly, the pigmentary particles under consideration in the invention are first formed from an intimate mixture of said pigments and from at least one polyol as binder.

According to one advantageous embodiment, the pigments under consideration and an effective amount of polyol(s) are milled.

The milling step requires the mixing of the pigments under consideration according to the invention with at least one polyol in an amount adjusted to give the resulting mixture firstly a good wetting quality, and secondly an adequate viscosity, and thus to facilitate the mechanical milling action. The milling uses standard apparatus and equipment.

According to one particular embodiment, the invention also contains at least one monoalcohol, especially as refreshing agent.

Besides its advantageous impact in terms of freshness agent, the presence of at least one monoalcohol moreover ensures better microbiological protection to the cosmetic formulation incorporating it.

This monoalcohol may be present in a composition according to the invention at up to 20% by weight thereof.

A composition according to the invention is advantageous in several respects.

Firstly, providing users with such a product allows them to gain access, via a single application, to the respective performance qualities of a care product and of a makeup composition, for example the makeup properties of a foundation and the sensory and/or efficacy properties of a very aqueous care product such as a care serum.

Moreover, such a product satisfies the expectations of potential users who are not satisfied at the present time with the sensory performance qualities manifested by the current makeup products.

Thus, as emerges from the text hereinbelow, and more particularly from the examples, the compositions according to the invention prove to be particularly advantageous with regard firstly to the sensory feeling that they give the user at the time of application, and secondly to the final makeup result obtained, which proves to be homogeneous and uniform.

During its uptake for application to the surface of a keratin material and more particularly the skin, the composition has a sufficiently thick texture to allow the user to take up the required amount in a single action. During the application to the surface of the keratin material, the texture of the composition breaks under the effect of the shear generated during its spreading by the user onto the surface of the keratin material, then affording an immediate fresh effect.

As regards the sensory feel, the compositions according to the invention prove to be soft to the touch, and have a slippery nature that makes them easy to apply and affords homogeneous distribution of all the pigments they contain onto the surface of the epidermis.

According to another of its aspects, a subject of the invention is also a process for making up and/or caring for a keratin material, in particular the skin, comprising at least one step that consists in applying to said keratin material a composition in accordance with the invention.

Cosmetic Composition

A composition according to the present invention as defined above has a minimum viscosity of 600 Pa·s at low shear, i.e. 0.010 s⁻¹, combined with a maximum viscosity of 0.8 Pa·s at high shear, i.e. 1000 s⁻¹.

These measurements are taken at 25° C. using a TA Instrument ARG2 imposed-stress rheometer equipped with a cone-plate measuring body fitted with an anti-evaporation device (bell jar). For each measurement, the sample is placed delicately in position and the measurements start 5 minutes after placing the sample in the jaws. The foundation is then subjected to a stress ramp from 10⁻³ to 10² at a set frequency of 1 Hz.

Water-Soluble or Water-Dispersible Gelling Agent

A composition of the invention comprises at least one water-soluble or water-dispersible gelling agent. This gelling agent may more particularly be chosen from acrylic polymers, polysaccharides, vinyl polymers, the sodium salts of polyhydroxycarboxylic acids, polyacrylamides, and mixtures thereof.

a—Hydrophilic Acrylic Polymers

According to the invention, the term “hydrophilic acrylic polymers” especially means non-hydrophobic and non-amphiphilic acrylic polymers.

Said hydrophilic acrylic polymers according to the invention are either polyacrylamidomethylpropanesulfonic acid (AMPS) acrylic polymers or acrylic acid polymers.

The presence of this hydrophilic acrylic polymer makes it possible especially to obtain a composition that has good stability properties.

Among the hydrophilic acrylic polymers that may be mentioned are the following polymers:

1) Acrylic Polymers Comprising at Least One Monomer Bearing a Sulfonic Group

According to a first embodiment, the hydrophilic acrylic polymer used according to the invention comprises at least one monomer bearing a sulfonic group.

The polymers used in accordance with the invention are homopolymers that may be obtained from at least one ethylenically unsaturated monomer bearing a sulfonic group, which may be in free form or partially or totally neutralized form.

Preferentially, the polymers in accordance with the invention are partially or totally neutralized with a mineral base (sodium hydroxide, potassium hydroxide or aqueous ammonia) or an organic base such as monoethanolamine, diethanolamine, triethanolamine, an aminomethylpropanediol, N-methylglucamine, basic amino acids, for instance arginine and lysine, and mixtures of these compounds. They are generally neutralized.

In the present invention, the term “neutralized” means polymers that are totally or virtually totally neutralized, i.e. at least 90% neutralized.

The polymers used in the composition of the invention generally have a number-average molecular weight ranging from 1000 to 20 000 000 g/mol, preferably ranging from 20 000 to 5 000 000 g/mol and even more preferentially from 100 000 to 1 500 000 g/mol.

These polymers according to the invention may be crosslinked or noncrosslinked.

The monomers bearing a sulfonic group of the polymer used in the composition of the invention are especially chosen from vinylsulfonic acid, styrenesulfonic acid, (meth)acrylamido(C₁-C₂₂)alkylsulfonic acids, N—(C₁-C₂₂)alkyl(meth)acrylamido(C₁-C₂₂)alkylsulfonic acids such as undecylacrylamidomethanesulfonic acid, and also partially or totally neutralized forms thereof, and mixtures thereof.

According to one preferred embodiment of the invention, the monomers bearing a sulfonic group are chosen from (meth)acrylamido(C₁-C₂₂)alkylsulfonic acids, for instance acrylamidomethanesulfonic acid, acrylamidoethanesulfonic acid, acrylamidopropanesulfonic acid, 2-acrylamido-2-methylpropanesulfonic acid, 2-methacrylamido-2-methylpropanesulfonic acid, 2-acrylamido-n-butanesulfonic acid, 2-acrylamido-2,4,4-trimethylpentanesulfonic acid, 2-methacrylamidododecylsulfonic acid and 2-acrylamido-2,6-dimethyl-3-heptanesulfonic acid, and also partially or totally neutralized forms thereof, and mixtures thereof.

More particularly, 2-acrylamido-2-methylpropanesulfonic acid (AMPS), and also partially or totally neutralized forms thereof, is used.

When the polymers are crosslinked, the crosslinking agents may be chosen from the polyolefinically unsaturated compounds commonly used for crosslinking polymers obtained by free-radical polymerization.

Examples of crosslinking agents that may be mentioned include divinylbenzene, diallyl ether, dipropylene glycol diallyl ether, polyglycol diallyl ethers, triethylene glycol divinyl ether, hydroquinone diallyl ether, ethylene glycol or tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl (meth)acrylate, allylic ethers of alcohols of the sugar series, or other allylic or vinyl ethers of polyfunctional alcohols, and also the allylic esters of phosphoric and/or vinylphosphonic acid derivatives, or mixtures of these compounds.

According to one preferred embodiment of the invention, the crosslinking agent is chosen from methylenebisacrylamide, allyl methacrylate and trimethylolpropane triacrylate (TMPTA). The degree of crosslinking generally ranges from 0.01 mol % to 10 mol % and more particularly from 0.2 mol % to 2 mol % relative to the polymer.

The homopolymer of monomers bearing a sulfonic group may be crosslinked with one or more crosslinking agents.

These homopolymers are generally crosslinked and neutralized, and they may be obtained according to the preparation process comprising the following steps:

(a) the monomer such as 2-acrylamido-2-methylpropanesulfonic acid in free form is dispersed or dissolved in a solution of tert-butanol or of water and tert-butanol;

(b) the monomer solution or dispersion obtained in (a) is neutralized with one or more mineral or organic bases, preferably aqueous ammonia NH₃, in the amount making it possible to obtain a degree of neutralization of the sulfonic acid functions of the polymer ranging from 90% to 100%;

(c) the crosslinking monomer(s) are added to the solution or dispersion obtained in (b);

(d) a standard free-radical polymerization is performed in the presence of free-radical initiators at a temperature ranging from 10 to 150° C.; the polymer precipitates in the tert-butanol-based solution or dispersion.

The preferred AMPS homopolymers are generally characterized in that they comprise, randomly distributed:

a) from 90% to 99.9% by weight of units of general formula (II) below:

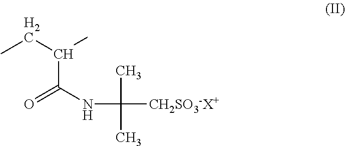

in which X⁺ denotes a proton, an alkali metal cation, an alkaline-earth metal cation or the ammonium ion, not more than 10 mol % of the cations X⁺ possibly being protons H⁺;

b) from 0.01% to 10% by weight of crosslinking units derived from at least one monomer containing at least two olefinic double bonds; the weight proportions being defined relative to the total weight of the polymer.

The homopolymers according to the invention that are more particularly preferred comprise from 98% to 99.5% by weight of units of formula (II) and from 0.2% to 2% by weight of crosslinking units.

A polymer of this type that may especially be mentioned is the crosslinked and neutralized 2-acrylamido-2-methylpropanesulfonic acid homopolymer sold by the company Clariant under the trade name Hostacerm AMPS (CTFA name: ammonium polyacryldimethyltauramide).

2) Acrylamide/AMPS Copolymers

According to another embodiment, the hydrophilic acrylic polymer is a crosslinked anionic copolymer formed from units derived from the reaction between (i) acrylamide (monomer 1), (ii) 2-acrylamido-2-methylpropanesulfonic acid (monomer 2, referred to hereinbelow for convenience as AMPS) and (iii) at least one polyolefinically unsaturated compound (monomer 3), constituting here the crosslinking agent.

The crosslinked anionic copolymers used in the context of the present invention are products that are already known per se and their preparation has been described especially in patent application EP-A-0 503 853, the content of which is consequently included in its entirety by reference in the present description.

The above copolymers may thus be obtained conventionally according to the emulsion polymerization technique from three different comonomers included in their constitution.

The polyolefinically unsaturated monomers used as crosslinking agents for the preparation of the copolymers in accordance with the invention are preferably chosen from the group formed by methylenebisacrylamide, allyl sucrose and pentaerythritol. Even more preferentially, use is made of methylenebisacrylamide.

Preferably, said polyolefinically unsaturated compound is present in the copolymer in a concentration of between 0.06 and 1 mmol per mole of the monomer units as a whole.

The ratio, expressed in mol %, between acrylamide and AMPS is preferentially between 85/15 and 15/85, advantageously between 70/30 and 30/70, even more preferentially between 65/35 and 35/65 and even more particularly between 60/40 and 40/60. In addition, AMPS is generally at least partially neutralized in the form of a salt, for example with sodium hydroxide, with potassium hydroxide or with a low molecular weight amine such as triethanolamine, or mixtures thereof.

A crosslinked copolymer that is particularly preferred in the context of the implementation of the present invention corresponds to the one prepared in Example 1 of patent application EP-A-0 503 853 mentioned above, and which is then in the form of a water-in-oil inverse emulsion. More precisely, this copolymer is formed from 60 mol % of acrylamide and 40 mol % of the sodium salt of AMPS, and it is crosslinked with methylenebisacrylamide in a proportion of 0.22 mmol per mole of the total monomer mixture. The final water-in-oil inverse emulsion preferably contains about 40% by weight of crosslinked copolymer as defined above and about 4% by weight of an ethoxylated fatty alcohol with an HLB of about 12.5.

Crosslinked copolymers that are more particularly used according to the invention are the products sold under the names Sepigel 305 (CTFA name: polyacrylamide/C13-14 isoparaffin/Laureth 7) or Simulgel 600 (CTFA name: acrylamide/sodium acryloyldimethyltaurate copolymer/isohexadecane/polysorbate 80) sold by the company SEPPIC, or Simulgel EG (CTFA name: sodium acrylate/sodium acryloyldimethyltaurate copolymer/isohexadecane/polysorbate 80).

3) Other Hydrophilic Acrylic Polymers

As other hydrophilic acrylic polymers that may be used according to the invention, mention may also be made of:
homopolymers or copolymers of acrylic or methacrylic acids or salts thereof and esters thereof, such as the products sold under the names Carbopol 934, 940, 954, 981 and 980 by the company Noveon, Synthalen L® from the company 3V, sodium polymethacrylate sold under the name Darvan No. 7® by the company Vanderbilt, the products sold under the names Versicol F or Versicol K by the company Allied Colloid, Ultrahold 8 by the company Ciba Geigy and polyacrylic acids of Synthalen K type,
polyacrylates and polymethacrylates such as glyceryl acrylate polymers, and in particular copolymers of glyceryl acrylate and of acrylic acid, such as the products sold under the names Lubrajel® MS, Lubrajel® CG, Lubrajel® DV, Lubrajel® NP, Lubrajel® OIL Lubrajel® Oil BG, Lubrajel® PF, Lubrajel® TW and Lubrajel® WA by the ® company Guardian Laboratories. Use is preferably made of Lubrajel MS,
polyacrylic acid/alkyl acrylate copolymers of Pemulen type,
copolymers of acrylic acid salt/vinyl alcohol, such as the product sold under the name Hydragen FN® from Cognis, and mixtures thereof

b—Amphiphilic Acrylic Polymers

Such polymers may be derived from the AMPS products described previously. These polymers comprise both a hydrophilic part and a hydrophobic part comprising at least one fatty chain. They are therefore amphiphilic polymers.

A fatty chain of such a polymer may comprise from 7 to 30 carbon atoms and in particular from 8 to 22 carbon atoms.

A hydrophobic AMPS copolymer in accordance with the invention may have a weight-average molecular weight ranging from 50 000 to 10 000 000, in particular from 100 000 to 8 000 000 and more particularly from 100 000 to 7 000 000.

A hydrophobic AMPS copolymer according to the invention may be crosslinked or noncrosslinked.

Among the crosslinking agents that may be suitable for use, mention may be made, in a nonlimiting manner, of methylenebisacrylamide, allyl methacrylate and trimethylolpropane triacrylate (TMPTA).

The degree of crosslinking may range from 0.01 mol % to 10 mol % and particularly from 0.2 mol % to 2 mol % relative to the polymer.

An amphiphilic AMPS polymer that is suitable for use in the invention may be chosen, for example, from statistical amphiphilic AMPS polymers modified by reaction with a C₆-C₂₂ n-monoalkylamine or di-n-alkylamine such as those described in patent application WO 00/31154.

These polymers may also contain other ethylenically unsaturated hydrophilic monomers chosen, for example, from acrylic acid, methacrylic acid or alkyl-substituted derivatives thereof or esters thereof obtained with mono- or polyalkylene glycols, acrylamide, methacrylamide, vinylpyrrolidone, itaconic acid and maleic acid, or mixtures thereof.

A polymer of the invention may be chosen from amphiphilic polymers of AMPS and of at least one ethylenically unsaturated monomer comprising at least one hydrophobic part containing from 7 to 30 carbon atoms, in particular from 8 to 22 carbon atoms and more particularly from 12 to 20 carbon atoms.

The hydrophobic part may be a saturated or unsaturated linear (for example n-octyl, n-decyl, n-hexadecyl, n-dodecyl or oleyl), branched (for example isostearic) or cyclic (for example cyclododecane or adamantane) alkyl radical.

An ethylenically unsaturated hydrophobic monomer that is suitable for use in the invention may be chosen from the acrylates or acrylamides of formula (1) below:

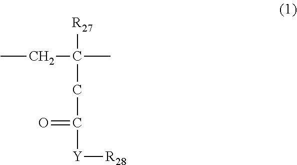

in which:
R₂₇ denotes a hydrogen atom or a linear or branched C₁-C₆ alkyl radical (preferably methyl),
Y denotes O or NH;
R₂₈ denotes a hydrophobic radical comprising a fatty chain containing from 7 to 30 carbon atoms, preferably from 8 to 22 and more particularly from 12 to 20 carbon atoms.

The hydrophobic radical R₂₈ is chosen from saturated or unsaturated linear C₇-C₂₂ alkyl radicals (for example n-octyl, n-decyl, n-hexadecyl, n-dodecyl or oleyl), branched alkyl radicals (for example isostearic) or cyclic alkyl radicals (for example cyclododecane or adamantane); C₇-C₁₈ alkylperfluoro radicals (for example the group of formula (CH₂)₂(CF₂)₉—CF₃); the cholesteryl radical or a cholesterol ester, for instance cholesteryl hexanoate; aromatic polycyclic groups, for instance naphthalene or pyrene.

Among these radicals, linear and branched alkyl radicals are more particularly preferred.

According to one embodiment of the invention, the hydrophobic radical R₂₈ may also comprise at least one alkylene oxide unit and in particular a polyoxyalkylene chain.

A polyoxyalkylene chain may be formed from ethylene oxide units and/or propylene oxide units and even more particularly be formed solely from ethylene oxide units.

The number of moles of oxyalkylene units may generally range from 1 to 30 mol, more particularly from 2 to 25 mol and even more particularly from 3 to 20 mol.

Among the AMPS amphiphilic polymers that are suitable for use in the invention, mention may be made of:
crosslinked or noncrosslinked, neutralized or non-neutralized copolymers comprising from 15% to 60% by weight of AMPS units and from 40% to 85% by weight of (C₈-C₁₆)alkyl(meth)acrylamide units or of (C₈-C₁₆)alkyl (meth)acrylate units relative to the polymer, such as those described in patent application EP-A-0 750 899;
terpolymers comprising from 10 mol % to 90 mol % of acrylamide units, from 0.1 mol % to 10 mol % of AMPS units and from 5 mol % to 80 mol % of n(C₆-C₁₈)alkylacrylamide units relative to the polymer, such as those described in U.S. Pat. No. 5,089,578.

As amphiphilic polymers that are suitable for use in the invention, mention may be made of polyoxyethylenated (crosslinked or noncrosslinked) copolymers of AMPS and of alkyl methacrylates, and mixtures thereof.

As amphiphilic polymers that are suitable for use in the invention, mention may also be made of copolymers of totally neutralized AMPS and of n-dodecyl, n-hexadecyl and/or n-octadecyl methacrylate, and also copolymers of AMPS and of n-dodecylmethacrylamide, which may be crosslinked or noncrosslinked.

Mention may also be made of crosslinked or noncrosslinked amphiphilic copolymers comprising, or even formed from:

(a) 2-acrylamido-2-methylpropanesulfonic acid (AMPS) units of formula (2) below:

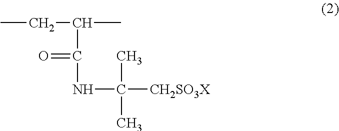

in which X may be a proton, an alkali metal cation, an alkaline-earth metal cation or an ammonium ion;

(b) and units of formula (3) below:

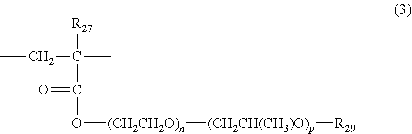

in which:
n and p, independently of each other, denote a number of moles and range from 0 to 30, in particular from 1 to 25 and more particularly from 3 to 20, with the proviso that n+p is less than or equal to 30, in particular less than 25 and more particularly less than 20;
R₂₇ has the same meaning indicated in the preceding formula (1), and
R₂₉ denotes a linear or branched alkyl radical comprising m carbon atoms, m ranging from 7 to 22 and preferably from 12 to 20.

In formula (2), the cation X may more particularly denote sodium or ammonium.

Mention may be made in particular of:

i. noncrosslinked polymers for which p=0, n=7 or 25, R₂₇ denotes methyl and R₂₉ represents a mixture of C₁₂-C₁₄ or C₁₆-C₁₈ alkyl, and

ii. crosslinked polymers for which p=0, n=8 or 25, R₂₇ denotes methyl and R₂₉ represents a mixture of C₁₆-C₁₈ alkyl.

These polymers are described and synthesized in document EP 1 069 142.

These particular amphiphilic polymers may be obtained according to the standard free-radical polymerization processes in the presence of one or more initiators such as, for example, azobisisobutyronitrile (AIBN), azobisdimethylvaleronitrile, 2,2-azobis[2-amidinopropane] hydrochloride (ABAH), organic peroxides such as dilauryl peroxide, benzoyl peroxide, tert-butyl hydroperoxide, etc., mineral peroxide compounds such as potassium persulfate or ammonium persulfate, or H₂O₂ optionally in the presence of reducing agents.

These amphiphilic polymers may be obtained by free-radical polymerization in tert-butanol medium, in which they precipitate.

By using precipitation polymerization in tert-butanol, it is possible to obtain a size distribution of the polymer particles that is particularly favorable for its uses.

The reaction may be performed at a temperature of between 0 and 150° C. and preferably between 10 and 100° C., either at atmospheric pressure or under reduced pressure.

The reaction may also be performed under inert atmosphere, and preferably under nitrogen.

A polymer in accordance with the invention may be partially or totally neutralized with a mineral or organic base such as those mentioned above.

The molar percentage concentration of the units of formula (2) and of the units of formula (3) in an amphiphilic polymer according to the invention may vary as a function of the desired cosmetic application, the nature of the emulsion (oil-in-water or water-in-oil emulsion) and the rheological properties of the desired formulation.

It can range between 0.1 and 99.9 mol %.

The molar proportion of units of formula (3) in an amphiphilic polymer according to the invention may preferably range from 0.1% to 50%, more particularly from 1% to 25% and even more particularly from 3% to 10%.

The molar proportion of units of formula (3) in an amphiphilic polymer according to the invention may preferably range from 50.1% to 99.9%, more particularly from 60% to 95% and even more particularly from 65% to 90%.

The distribution of the monomers in the polymers of the invention may be, for example, alternate, block (including multiblock) or random.

As a guide, and without this being limiting, mention may be made of the following commercial references: Aristoflex® HMS and Aristoflex® HMB sold by Clariant, these two references relating to crosslinked polymers.

Aristoflex® HMS is the name of the AMPS/ethoxylated (25 EO) cetearyl methacrylate copolymer 80/20, crosslinked with trimethylolpropane triacrylate (TMPTA) or ammonium acryloyldimethyltaurate/stearate-25 methacrylate crosspolymer as the INCI name.

As the INCI name, Aristoflex® HMB is ammonium acryloyldimethyltaurate/Beheneth-25 methacrylate crosspolymer.

It is also possible to use, as hydrophobic AMPS copolymer, noncrosslinked AMPS copolymers (Aristoflex® LNC or SNC), which are also effective in terms of stabilizing emulsions. On the other hand, the textures then obtained are less novel, especially as regards the water transformation effect.

As the INCI name, Aristoflex® LNC is ammonium acryloyldimethyltaurate/Laureth-7 methacrylate copolymer.

As the INCI name, Aristoflex® SNC is ammonium acryloyldimethyltaurate/Steareth-8 methacrylate copolymer.

Among the acrylic polymers that may be combined with a combination in accordance with the invention, mention may also be made of neutralized crosslinked acrylic homopolymers or copolymers.

c—Neutralized Crosslinked Acrylic Homopolymers or Copolymers

All crosslinked acrylic homopolymers or copolymers are suitable for use in the present invention provided that they are used in an at least partially neutralized form.

As regards these crosslinked acrylic polymers already neutralized before use, or otherwise, examples that may be mentioned include:
Cosmedia SP® or crosslinked sodium polyacrylate containing 90% solids and 10% water, Cosmedia SPL® or sodium polyacrylate as an inverse emulsion containing about 60% dry active material, an oil (hydrogenated polydecene) and a surfactant (PPG-5 laureth-5), both sold by the company Cognis,
modified or unmodified carboxyvinyl polymers (for instance optionally crosslinked acrylic acid polymers), such as the products sold under the names Carbopol (INCI name: Carbomer) by the company Goodrich,
partially neutralized crosslinked sodium polyacrylates that are in the form of an inverse emulsion comprising at least one polar oil, for example the product sold under the name Luvigel® EM sold by the company BASF, and
mixtures thereof.

d—Polysaccharides

As polysaccharides that are suitable for use in the invention, mention may be made especially of a polysaccharide biopolymer, for instance the commercial reference Fucogel 1.5P from Solabia (fucose-rich polysaccharide (20%) at 1.1% in stabilized water (1.5% phenoxyethanol)), xanthan gum, guar gum, locust bean gum, acacia gum, karaya gum, sandarac gum, shellac resin, dammar resins, elemi gums, copal resins, scleroglucans, anionic, cationic, amphoteric or nonionic chitin and chitosan derivatives, carrageenans, gellans, alginates, inulins, celluloses, glycoaminoglycans, mucopolysaccharides such as hyaluronic acid, and chondroitin sulfates, and mixtures thereof.

Among the celluloses, mention may be made of microcrystalline cellulose, a cellulose polymer such as hydroxyethylcellulose, hydroxymethylcellulose, hydroxypropylcellulose, methylcellulose, ethylhydroxyethylcellulose or carboxymethylcellulose, or a quaternized cellulose derivative.

According to one preferred mode of the invention, the water-soluble or water-dispersible gelling agents are chosen from:
modified or unmodified carboxyvinyl polymers,
polyacrylates and polymethacrylates,
optionally crosslinked and/or neutralized 2-acrylamido-2-methylpropanesulfonic acid polymers and copolymers,
ethoxylated (25 EO) AMPS/stearyl methacrylate copolymers crosslinked with trimethylolpropane triacrylate (TMPTA),
AMPS/acrylamide copolymers,
polyacrylamides,
associative polyurethanes,
polysaccharides such as xanthan gum, xanthan derivatives, carrageenans, gellans, alginates, celluloses such as microcrystalline cellulose and cellulose polymers such as hydroxyethylcellulose, hydroxymethylcellulose, hydroxypropylcellulose, methylcellulose, ethylhydroxyethylcellulose and carboxymethylcellulose,
and mixtures thereof.

The associative polyurethanes can for example be chosen among the C16-OE120-C16 polymer from the company Servo Delden (sold under the name SER AD FX1100, molecule bearing a urethane function and having a weight-average molecular weight of 1300), OE being an oxyethylene unit, Rheolate 205 bearing a urea function sold by the company Rheox, or Rheolate 208 or 204 (these polymers being sold in pure form) or DW 1206B from Rohm & Haas bearing a C₂₀ alkyl chain and a urethane bond, sold at 20% solids in water. It is also possible to use solutions or dispersions of these associative polyurethanes, especially in water or in aqueous-alcoholic medium. Examples of such polymers that may be mentioned include SER AD FX1010, SER AD FX1035 and SER AD 1070 from the company Servo Delden, Rheolate 255, Rheolate 278 and Rheolate 244 sold by the company Rheox. It is also possible to use the products DW 1206F and DW 1206J, and also Acrysol RM 184 or Acrysol 44 from the company Rohm & Haas, or alternatively Borchigel LW 44 from the company Borchers,

Optionally crosslinked and/or neutralized 2-acrylamido-2-methylpropanesulfonic acid polymers and copolymers or ethoxylated AMPS/stearyl methacrylate copolymers (25 EO) crosslinked with trimethylolpropane triacrylate (TMPTA) prove to be particularly advantageous.

The water-soluble or water-dispersible gelling agents may be present in the compositions according to the invention in a content ranging from 0.05% to 40% by weight, preferably from 0.1% to 20% and better still from 0.2% to 15% by weight relative to the total weight of the composition.

Pigments

As stated previously, the pigments under consideration in a composition according to the invention in the form of individualized pigmentary particles, i.e. in non-aggregated or sparingly aggregated form, which is stabilized over time.

The term “individualized pigmentary particle” means a solid particulate material with a mean particle size (after milling) of between 0.01 μm and 100 μm, in particular between 0.05 and 10 μm and especially between 0.1 and 1 μm.

The term “pigments” should be understood as meaning white or colored, mineral or organic particles that are untreated and are insoluble in an aqueous solution, which are intended to color and/or opacify the makeup film containing them.

The composition according to the invention may contain from 2% to 20% by weight, and in particular from 3% to 15% by weight of pigments relative to the total weight of said composition.

As mineral pigments that may be used in the invention, mention may be made of titanium, zirconium or cerium oxides or dioxides, and also zinc oxide, iron oxide or chromium oxide, ferric blue, manganese violet, ultramarine blue and chromium hydrate, and untreated mixtures thereof.

Advantageously, the pigments in accordance with the invention are iron oxides and/or titanium dioxides.

According to the invention, the pigmentary particles are formed solely from pigments and polyols.

According to one embodiment, the pigmentary particles may be formed from pigments, polyols and water.

The presence of water may, in fact, prove to be advantageous for gaining access to a pigments/polyol mixture that is more blendable and thus easier to manipulate during the subsequent milling step.

In general, water, if present, is used in a proportion of not more than 80% expressed relative to the total polyol+water weight of this pigmentary preparation.

Polyol

A composition according to the invention also comprises at least one polyol.

For the purposes of the present invention, the term “polyol” should be understood as meaning any organic molecule comprising at least two free hydroxyl groups.

A polyol that is suitable for use in the invention may be a compound of linear, branched or cyclic, saturated or unsaturated alkyl type, bearing on the alkyl chain at least two —OH functions and in particular at least three —OH functions.

The polyols that are suitable for formulating a composition according to the present invention are those especially containing from 3 to 12 carbon atoms, and preferably 3 to 8 carbon atoms.

According to one particular mode of the invention, the polyol is a compound of linear, branched or cyclic, saturated or unsaturated alkyl type, bearing on the alkyl chain at least three —OH functions.

According to one embodiment, a composition of the invention may comprise a mixture of polyols.

According to one preferred mode of the invention, said polyol is in liquid form at room temperature.

Advantageously, the polyol may be chosen, for example, from ethylene glycol, propylene glycol, 1,3-propanediol, butylene glycol, isoprene glycol, pentylene glycol, hexylene glycol, glycerol, polyglycerols, such as glycerol oligomers, for instance diglycerol, and mixtures thereof.

According to one preferred mode of the invention, said polyol is chosen from glycerol, sorbitol, xylose, trehalose and maltose, and mixtures thereof.

According to a more particularly preferred mode of the invention, said polyol is glycerol.

A polyol that is suitable for use in the invention may advantageously be natural or of natural origin.

A composition of the invention may comprise from 0.5% to 15% by weight, preferably from 1% to 14% by weight and better still from 2% to 12% by weight of polyol(s) relative to the total weight of said composition.

As stated hereinabove, a composition according to the invention comprises at least one polyol in a polyol(s)/pigments weight ratio ranging from 0.025 to 7.5, preferably from 0.05 to 7 and more particularly from 0.1 to 6.

More particularly, the content of pigment(s) in the pigmentary particles is advantageously between 40% and 70% by weight and the polyol is advantageously between 30% and 60% by weight relative to the total weight of said particles.

Thus, advantageously, pigmentary particles comprising 40% to 70% by weight of pigments and 5% to 7% by weight of glycerol, relative to the total weight of said pigmentary particles, may be used according to the invention.

Aqueous Chase

As stated hereinabove, a composition comprises at least one aqueous phase.

In a composition according to the invention, the aqueous phase is present in a proportion of at least 65% by weight, in particular from 65% to 90% by weight and better still from 66% to 85% by weight of aqueous phase relative to the total weight of the composition.

For the purposes of the invention, the amount of aqueous phase takes into account the amount of polyol.

The aqueous phase of the composition according to the invention comprises water and, where appropriate, at least one water-soluble solvent.

In the present invention, the term “water-soluble solvent” denotes a compound that is liquid at room temperature and water-miscible (miscibility with water of greater than 50% by weight at 25° C. and atmospheric pressure).

The water-soluble solvents that may be used in the compositions according to the invention may also be volatile.

Among the water-soluble solvents, other than the abovementioned polyols, which may be used in the second composition in accordance with the invention, mention may be made especially of lower monoalcohols containing from 1 to 5 carbon atoms, such as ethanol and isopropanol, C₃ and C₄ ketones and C₂-C₄ aldehydes.

In order to ensure the fresh sensation of the product on application, and also a satisfactory makeup result, the composition may advantageously contain at least one monoalcohol, especially a C₂ to C₈ lower monoalcohol, in a proportion of from 0 to 20% by weight relative to its total weight. The presence of a monoalcohol is advantageous in several respects. It contributes toward the sensation of freshness on application and improves the microbiological protection of the composition containing it.

The lower monoalcohols that are more particularly suitable for use in the invention comprise from 2 to 8 carbon atoms, especially from 2 to 6 carbon atoms and in particular from 2 to 4 carbon atoms, for instance ethanol, isopropanol, propanol and butanol.

Ethanol and isopropanol are thus most particularly suitable for use in the invention, preferably ethanol.

According to one particular variant of the invention, a composition according to the invention may have an aqueous phase/pigments weight ratio that may range from 3 to 45, preferably from 4 to 35 and more particularly from 5 to 25.

The aqueous phase may also comprise any water-soluble or water-dispersible compound, other than a polyol, that is compatible with an aqueous phase, such as gelling agents, film-forming polymers, thickeners or surfactants especially as defined hereinbelow, and mixtures thereof.

Fatty Substances

The composition may also comprise one or more fatty substances other than the abovementioned polyols, if present.

Thus, a composition according to the invention may contain from 0 to 15% by weight of fatty substance relative to its total weight.

Specifically, the fatty substances may afford emollience and may improve the application qualities of the product and the final feel of the made-up skin.

Said fatty substance may especially be at least one oil as mentioned hereinbelow.

The term “oil” means any fatty substance that is in liquid form at room temperature and atmospheric pressure.

A composition of the invention may comprise a content of less than 15% by weight, preferably less than 10% by weight, in particular less than 8% by weight and better still less than 5% by weight of liquid fatty phase(s), relative to the total weight of said composition.

An oily phase that is suitable for preparing the cosmetic compositions according to the invention may comprise hydrocarbon-based oils, silicone oils, fluoro oils or non-fluoro oils, or mixtures thereof.

The oils are preferably nonvolatile.

They may be of animal, plant, mineral or synthetic origin. According to one embodiment variant, oils of plant origin are preferred.

For the purposes of the present invention, the term “non-volatile oil” means an oil with a vapor pressure of less than 0.13 Pa.

For the purposes of the present invention, the term “silicone oil” means an oil comprising at least one silicon atom, and especially at least one Si—O group.

The term “fluoro oil” means an oil comprising at least one fluorine atom.

The term “hydrocarbon-based oil” means an oil mainly containing hydrogen and carbon atoms.

The oils may optionally comprise oxygen, nitrogen, sulfur and/or phosphorus atoms, for example in the form of hydroxyl or acid radicals.

For the purposes of the invention, the term “volatile oil” means any oil that is capable of evaporating on contact with keratin materials in less than one hour, at room temperature and atmospheric pressure. The volatile oil is a volatile cosmetic compound, which is liquid at room temperature, especially having a nonzero vapor pressure, at room temperature and atmospheric pressure, in particular having a vapor pressure ranging from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1,300 Pa (0.01 to 10 mmHg).

Volatile Oils

The volatile oils may be hydrocarbon-based oils or silicone oils.

Among the volatile hydrocarbon-based oils containing from 8 to 16 carbon atoms, mention may be made especially of branched C₈-C₁₆ alkanes, for instance C₈-C₁₆ isoalkanes (also known as isoparaffins), isododecane, isodecane, isohexadecane and, for example, the oils sold under the trade names Isopar or Permethyl, branched C₈-C₁₆ esters, for instance isohexyl neopentanoate, and mixtures thereof. Preferably, the volatile hydrocarbon-based oil is chosen from volatile hydrocarbon-based oils containing from 8 to 16 carbon atoms and mixtures thereof, in particular from isododecane, isodecane and isohexadecane, and is especially isododecane.

Mention may also be made of linear volatile alkanes comprising from 8 to 16 carbon atoms, in particular from 10 to 15 carbon atoms and more particularly from 11 to 13 carbon atoms, with a flash point in the range from 30 to 120° C. and more particularly from 40 to 100° C. In particular, the flash point is in the range from 70 to 120° C. and more particularly from 80 to 100° C., and especially about 89° C. The flash point is in particular measured according to standard ISO 3679.

A volatile linear alkane that is suitable for use in the invention may advantageously be of plant origin.

Such an alkane may be obtained, directly or in several steps, from a plant starting material such as an oil, a butter, a wax, etc. As examples of alkanes that are suitable for use in the invention, mention may be made of the alkanes described in the patent application from the company Cognis WO 2007/068 371, or WO 2008/155 059 (mixtures of different alkanes differing by at least one carbon).

As examples of linear alkanes that are suitable for use in the invention, mention may be made of n-nonane (C9), n-decane (C10), n-undecane (C11), n-dodecane (C12), n-tridecane (C13), n-tetradecane (C14), n-pentadecane (C15), n-hexadecane (C16) and n-heptadecane (C17), and mixtures thereof, and in particular mixtures of n-undecane (C11) and n-tridecane (C13) obtained in Examples 1 and 2 of patent application WO 2008/155 059 from the company Cognis, such as the mixture of n-undecane (C11) and n-tridecane (C13).

More particularly, a volatile linear alkane that is suitable for use in the invention may be used in the form of an n-undecane/n-tridecane mixture.

Volatile silicone oils that may be mentioned include linear volatile silicone oils such as hexamethyldisiloxane, octamethyltrisiloxane, decamethyltetrasiloxane, tetradecamethylhexasiloxane and hexadecamethylheptasiloxane.

Volatile cyclic silicone oils that may be mentioned include hexamethylcyclotrisiloxane, octamethylcyclotetrasiloxane, decamethylcyclopentasiloxane and dodecamethylcyclohexasiloxane.

A composition of the invention may comprise from 0.1% to 10% by weight and preferably from 0.5% to 5% by weight of volatile oil(s) relative to the total weight of said composition.

Nonvolatile Oils

The nonvolatile oils may be chosen especially from nonvolatile hydrocarbon-based, fluoro and/or silicone oils.

Nonvolatile hydrocarbon-based oils that may especially be mentioned include:
hydrocarbon-based oils of animal origin,
hydrocarbon-based oils of plant origin, such as phytostearyl esters, such as phytostearyl oleate, phytostearyl isostearate and lauroyl/octyldodecyl/phytostearyl glutamate (Ajinomoto, Eldew PS203), triglycerides formed from fatty acid esters of glycerol, in particular in which the fatty acids may have chain lengths ranging from C₄ to C₃₆ and especially from C₁₈ to C₃₆, these oils possibly being linear or branched, and saturated or unsaturated; these oils may especially be heptanoic or octanoic triglycerides, shea oil, alfalfa oil, poppy oil, millet oil, barley oil, quinoa oil, rye oil, candlenut oil, passionflower oil, shea butter, aloe vera oil, sweet almond oil, peach stone oil, groundnut oil, argan oil, avocado oil, baobab oil, borage oil, broccoli oil, calendula oil, camellina oil, canola oil, carrot oil, safflower oil, flax oil, rapeseed oil, cotton oil, coconut oil, marrow seed oil, wheatgerm oil, jojoba oil, lily oil, macadamia oil, corn oil, meadowfoam oil, St John's Wort oil, monoi oil, hazelnut oil, apricot kernel oil, walnut oil, olive oil, evening primrose oil, palm oil, blackcurrant pip oil, kiwi seed oil, grapeseed oil, pistachio oil, winter squash oil, pumpkin oil, quinoa oil, musk rose oil, sesame oil, soybean oil, sunflower oil, castor oil and watermelon oil, and mixtures thereof, or alternatively caprylic/capric acid triglycerides, such as those sold by the company Stearineries Dubois or those sold under the names Miglyol 810®, 812® and 818® by the company Dynamit Nobel;
synthetic ethers containing from 10 to 40 carbon atoms, such as dicaprylyl ether;
synthetic esters, for instance the oils of formula R₁COOR₂, in which R₁ represents a linear or branched fatty acid residue containing from 1 to 40 carbon atoms and R₂ represents a hydrocarbon-based chain, which is especially branched, containing from 1 to 40 carbon atoms, on condition that R₁+R₂ 10. The esters may be chosen especially from esters of alcohol and of fatty acid, for instance cetostearyl octanoate, esters of isopropyl alcohol, such as isopropyl myristate, isopropyl palmitate, ethyl palmitate, 2-ethylhexyl palmitate, isopropyl stearate, octyl stearate, hydroxylated esters, for instance isostearyl lactate, octyl hydroxystearate, alcohol or polyalcohol ricinoleates, hexyl laurate, neopentanoic acid esters, for instance isodecyl neopentanoate, isotridecyl neopentanoate, and isononanoic acid esters, for instance isononyl isononanoate and isotridecyl isononanoate.
polyol esters and pentaerythritol esters, for instance dipentaerythritol tetrahydroxystearate/tetraisostearate,
fatty alcohols that are liquid at room temperature, with a branched and/or unsaturated carbon-based chain containing from 12 to 26 carbon atoms, for instance 2-octyldodecanol, isostearyl alcohol and oleyl alcohol;
C₁₂-C₂₂ higher fatty acids, such as oleic acid, linoleic acid and linolenic acid, and mixtures thereof;
dialkyl carbonates, the two alkyl chains possibly being identical or different, such as dicaprylyl carbonate sold under the name Cetiol CC® by Cognis; and
oils of high molar mass, in particular with a molar mass ranging from about 400 to about 2000 g/mol and in particular from about 650 to about 1600 g/mol. As oils of high molar mass that may be used in the present invention, mention may be made especially of linear fatty acid esters with a total carbon number ranging from 35 to 70, for instance pentaerythrityl tetrapelargonate, hydroxylated esters, such as polyglyceryl-2 triisostearate, aromatic esters, such as tridecyl trimellitate, esters of branched C₂₄-C₂₈ fatty alcohols or fatty acids, such as those described in U.S. Pat. No. 6,491,927, and pentaerythritol esters, and especially triisoarachidyl citrate, glyceryl triisostearate, glyceryl tris(2-decyl)tetradecanoate, polyglyceryl-2 tetraisostearate or pentaerythrityl tetrakis(2-decyl)tetradecanoate; phenyl silicones, such as Belsil PDM 1000 from the company Wacker (MM=9000 g/mol), non-volatile polydimethylsiloxanes (PDMS), PDMSs comprising alkyl or alkoxy groups that are pendent and/or at the end of the silicone chain, these groups each containing from 2 to 24 carbon atoms, phenyl silicones, for instance phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates, dimethicones or phenyl trimethicones with a viscosity of less than or equal to 100 cSt, and mixtures thereof; and also mixtures of these various oils.

A composition of the invention may comprise from 0.1% to 10% by weight and preferably from 0.5% to 5% by weight of nonvolatile oil(s) relative to the total weight of said composition.

Particles and Other Compounds

According to one particular embodiment, a cosmetic composition of the invention may comprise, besides the pigmentary particles as mentioned previously, water-soluble dyestuffs, or even particles in free form.

Water-Soluble Dyestuff

For the purposes of the invention, the term “water-soluble dyestuff” means any natural or synthetic, generally organic compound, which is soluble in an aqueous phase or water-miscible solvents and which is capable of coloring.

As water-soluble dyes that are suitable for use in the invention, mention may be made especially of synthetic or natural water-soluble dyes, for instance FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, FDC Blue 1, betanine (beetroot), carmine, copper chlorophylline, methylene blue, anthocyanins (enocianin, black carrot, hibiscus and elder), caramel and riboflavin.

For the purposes of the present invention, the additional particles are more particularly chosen from:
fillers;
nacres;
particles with a metallic tint; and
mixtures thereof.

These particles may or may not be treated with a surface agent.

Fillers

The term “filler” should be understood as meaning colorless or white, solid particles of any form, which are in a form that is insoluble and dispersed in the medium of the composition. These fillers, of mineral or organic, natural or synthetic nature, give the composition softness and give the makeup result a matt effect and uniformity.

The fillers used in the compositions according to the present invention may be of lamellar, globular or spherical form, fibres or in any form intermediate between these defined forms.

Among the mineral fillers that may be used in the compositions according to the invention, mention may be made of natural or synthetic mica, natural or synthetic sericite, silica, hydroxyapatite, boron nitride, hollow silica microspheres (Silica beads from Maprecos), glass or ceramic microcapsules; composites of silica and titanium dioxide, such as the TSG series sold by Nippon Sheet Glass, and mixtures thereof.

Among the organic fillers that may be used in the compositions according to the invention, mention may be made of starch-based or cellulose-based powders. Examples of such fillers that may be mentioned include the Dry Flo products sold by Akzo Nobel and the Cellubeads products sold by the company Daito Kasei.

Advantageously, the fillers in accordance with the invention are mineral fillers, preferably chosen from mica, sericite, silica, glass particles and boron nitride, and mixtures thereof.

Nacres

The term “nacres” should be understood as meaning iridescent or non-iridescent colored particles of any form, especially produced by certain molluscs in their shell, or else synthesized, and which have a color effect by optical interference.

The nacres may be chosen from nacreous pigments such as titanium mica coated with an iron oxide, titanium mica coated with bismuth oxychloride, titanium mica coated with chromium oxide, titanium mica coated with an organic dye and also nacreous pigments based on bismuth oxychloride. They may also be mica particles at the surface of which are superposed at least two successive layers of metal oxides and/or of organic dyestuffs.

Examples of nacres that may also be mentioned include natural mica coated with titanium oxide, with iron oxide, with natural pigment or with bismuth oxychloride.

The nacres may more particularly have a yellow, pink, red, bronze, orange, brown, gold and/or coppery color or tint.

Advantageously, the nacres in accordance with the invention are micas coated with titanium dioxide or with iron oxide, and also bismuth oxychloride.

Particles with a Metallic Tint

The term “particles with a metallic tint”, within the meaning of the present invention, denotes particles whose nature, size, structure and surface state allow them to reflect the incident light, especially in a non-iridescent manner.

Particles with a substantially flat outer surface are also suitable, since they can, if their size, structure and surface state allow it, more easily give rise to a strong specular reflection, which may then be termed a mirror effect.

The particles with a metallic tint that may be used in the invention may, for example, reflect light in all the components of the visible region without significantly absorbing one or more wavelengths. The spectral reflectance of these particles may, for example, be greater than 70% and better still at least 80%, or even 90% or 95%, in the range 400-700 nm.

These particles generally have a thickness of less than or equal to 1 μm, especially less than or equal to 0.7 μm and in particular less than or equal to 0.5 μm.

The particles with a metallic tint that may be used in the invention are chosen in particular from:
particles of at least one metal and/or of at least one metal derivative,
particles comprising a monomaterial or multimaterial organic or mineral substrate, at least partially coated with at least one layer with a metallic tint comprising at least one metal and/or at least one metal derivative, and
mixtures of said particles.

Among the metals that may be present in said particles, mention may be made, for example, of Ag, Au, Cu, Al, Ni, Sn, Mg, Cr, Mo, Ti, Zr, Pt, Va, Rb, W, Zn, Ge, Te and Se, and mixtures or alloys thereof. Ag, Au, Cu, Al, Zn, Ni, Mo and Cr and mixtures or alloys thereof (for example bronzes and brasses) are preferred metals.

The term “metal derivatives” is intended to denote compounds derived from metals, especially oxides, fluorides, chlorides and sulfides.

Among the metal derivatives that may be present in said particles, mention may be made especially of metal oxides, for instance titanium oxide, especially TiO₂, iron oxide, especially Fe₂O₃, tin oxide, chromium oxide, barium sulfate and the following compounds: MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅, MoS₂, and mixtures or alloys thereof.

Mention may be made of glass particles coated with a metallic layer, especially those described in documents JP-A-09188830, JP-A-10158450, JP-A-10158541, JP-A-07258460 and JP-A-05017710.

The particles with a metallic tint may also be chosen from particles formed from a stack of at least two layers with different refractive indices. These layers may be of polymeric or metallic nature and may especially include at least one polymer layer.

Thus, the particles with a metallic effect may be particles derived from a multilayer polymer film.

The choice of materials intended to constitute the various layers of the multilayer structure is obviously made so as to give the particles thus formed the desired metallic effect.

Such particles are especially described in WO 99/36477, U.S. Pat. No. 6,299,979 and U.S. Pat. No. 6,387,498 and more particularly identified below in the goniochromatic section.

Dispersant

Advantageously, a composition according to the invention may also comprise a dispersant.

Such a dispersant may be a surfactant, an oligomer, a polymer or a mixture of several thereof.

According to one particular embodiment, a dispersant in accordance with the invention is a surfactant.

As dispersants that are suitable for use in the invention, mention may be made especially of surfactants with high hydrophilicity, with an HLB of greater than 10, preferably greater than 13 and more particularly greater than 15.

The term “HLB of greater than or equal to 10” means a surfactant having, at 25° C., an HLB balance (hydrophilic-lipophilic balance), within the Griffin meaning, of greater than or equal to 10.

The Griffin HLB value is defined in J. Soc. Cosm. Chem. 1954 (volume 5), pages 249-256.

Reference may also be made to Kirk-Othmer's Encyclopedia of Chemical Technology, volume 22, pp. 333-432, 3^{rd} edition, 1979, Wiley, for the definition of the (emulsifying) properties and functions of surfactants, in particular pp. 347-377 of this reference, for anionic and nonionic surfactants.

Reference may also be made to surfactants with an HLB of greater than or equal to 10 that are cited in McCutcheon's Emulsifiers & Detergents, International edition of 1998 et seq. Examples that may be mentioned are those given on pages 223 to 231 of the HLB Index section of the 1998 edition.

A surfactant that is suitable for use in the invention may be chosen from ionic, anionic, cationic and nonionic surfactants, and mixtures thereof. The surfactant is preferably nonionic.

The nonionic dispersant may be chosen from polyoxyethylene glycol ethers or esters (POE/PEG ethers or esters) or polyoxypropylene glycol ethers or esters (PPG ethers or esters), from sugar ethers or esters, from glycerol or polyglycerol ethers or esters and from ethoxylated glyceride esters (POE glyceryl esters) or from mixtures thereof.

The POE ethers according to the invention may be, for example, PEG-40 hydrogenated castor oil, PEG-60 hydrogenated castor oil, PEG-40 stearate, jojoba wax PEG-120 ester, PEG-100 stearate or PEG-100 isostearate.

The sugar esters according to the invention may be, for example, sucrose laurate, sucrose palmitate, sucrose myristate, sucrose stearate or lauryl glucoside.

The ethoxylenated glycerides according to the invention may be chosen from PEG-80 glyceryl soyate, PEG-20 glyceryl stearate, PEG-60 almond glycerides, PEG-42 babassu glycerides, PEG-60 evening primrose glycerides, PEG-60 corn glycerides, PEG-45 palm kernel glycerides, PEG-60 maracuja glycerides or PEG-192 apricot kernel glycerides.

The nonionic dispersant is preferably a polyoxyethylenated ether, preferably with an HLB value of greater than 15. This POE ether may be chosen, for example, from ceteareth-20, steareth-20, oleth-20, beheneth-25, steareth-21, ceteareth-25 and the oxyethylenated ether of cetearyl alcohol containing 25 oxyethylene groups, C12-13 pareth-23, oleth-30, laureth-23 or ceteareth-30.

The anionic dispersant may be chosen from sulfuric acid esters (sodium lauryl sulfate, sodium cetearyl sulfate), phosphoric acid esters (laureth-4 phosphate) and sarcosine derivatives (lauroyl sarcosine).

Active Agents

As active agents that may be used in the composition of the invention, examples that may be mentioned include moisturizers, such as protein hydrolysates and polyols, for instance glycerol, glycols such as polyethylene glycols, and sugar derivatives; natural extracts; vitamins, such as vitamin A (retinol), vitamin E (tocopherol), vitamin C (ascorbic acid), vitamin B5 (panthenol), vitamin B3 (niacinamide), derivatives of these vitamins (especially esters) and mixtures thereof; desquamating agents; urea; caffeine; salicylic acid and derivatives thereof; α-hydroxy acids such as lactic acid and glycolic acid and derivatives thereof; retinoids such as carotenoids and vitamin A derivatives; sunscreens; extracts of algae, fungi, plants, yeasts or bacteria; enzymes; tensioning agents and agents acting on the capillary circulation, such as manganese gluconate (Givobio GMn® from SEPPIC), extract of lupin (Eclaline® from Silab), extract of bitter orange blossom (Remoduline® from Silab), extracts of Ruscus, of common horse chestnut, of ivy, of ginseng and of melilot, caffeine, an extract of black tea such as Kombuchka from Sederma; rutin salts; an extract of Corallina officinalis such as the product sold by CODIF; and mixtures thereof.

The sunscreens (or UV-screening agents) may be chosen from organic screening agents and physical screening agents, and mixtures thereof.

The chemical sunscreens that may be used in the composition of the invention may comprise all the UVA and UVB screening agents that may be used in the field of cosmetics.

Additives

A cosmetic composition according to the invention may also comprise any additive usually used in the field concerned, chosen, for example, from film-forming polymers, antioxidants, essential oils, preserving agents, fragrances, neutralizers and antiseptic agents, and mixtures thereof.

Needless to say, a person skilled in the art will take care to select the optional adjuvant(s) to be added to the composition according to the invention, such that the advantageous properties intrinsically associated with the composition in accordance with the invention are not, or are not substantially, adversely affected by the envisioned addition.

These adjuvants are generally present in the composition in a proportion ranging from 0.01% to 20% by weight and preferably from 0.1% to 10% by weight relative to the total weight of said composition.

According to one embodiment, a composition of the invention may advantageously be in the form of a foundation.

A composition of the invention may be obtained via any preparation process known to those skilled in the art.

The pigments used according to the present invention, i.e. in the form of pigmentary particles comprising at least one polyol, are obtained according to the following process:
premixing the pigments and the polyol(s) (partly or the entire amount) and optionally an adjusted amount of water, so as to obtain a preground material of adequate texture;
treating (one or more times depending on the desired milling quality) this premix in a continuous or batch three-roll mill or ball mill, so as to obtain the pigmentary particles according to the invention.

It is advantageous to mill them so as to ensure the fineness and homogeneity of distribution of the pigments in the formulation, in order to optimize the expression of the color of each pigment in a formulation and also to optimize its stability (stability of the color and sedimentation).

The pigmentary particles thus obtained are then introduced into the aqueous phase with the other ingredients, generally with vigorous stirring.

The examples that follow are presented as nonlimiting illustrations of the field of the invention.

### EXAMPLE 1

| Chemical name | weight % |
|---|---|
| Silica microspheres | 1.5 |
| Titanium oxide (untreated anatase) (CI: 77891) | 4.68 |
| Red iron oxide (CI: 77491) | 0.34 |
| Yellow iron oxide (CI: 77492) | 0.86 |
| Black iron oxide (CI: 77499) | 0.12 |
| Methyl p-hydroxybenzoate | 0.4 |
| Isohexadecane | 2 |
| 80/20 AMPS/ethoxylated (25 EO) stearyl methacrylate copolymer | 0.7 |
| crosslinked with trimethylolpropane triacrylate (TMPTA) | |
| (Aristoflex HMS from Clariant) | |
| Methylsilanol/silicate crosspolymer | 1.5 |
| Denatured absolute ethyl alcohol | 5 |
| Microbiologically clean deionized water | qs 100 |
| Glycerol | 7 |

The composition is prepared according to the following protocol:
Premix the pigments and the glycerol and advantageously an adjusted amount of water, so as to obtain a preground material of adequate texture.
Treat (one or more times depending on the desired milling quality) this premix in a continuous or batch three-roll mill or ball mill, so as to obtain pigmentary particles (ground pigmentary material).
In parallel, boil the water, incorporate therein at appropriate temperatures of various water-soluble ingredients (preserving agents, alcohol, active agents), the surfactants and the oils.
With vigorous stirring, for example using a Rayneri blender, incorporate the gelling agent(s)/texturing agent(s) (AMPS).
Next, with continued stirring, incorporate the pigmentary particles (the ground pigmentary material) and the rest of the ingredients.

The product thus obtained has, on application to facial skin, a fluid, light, very slippery texture with a sensation of water that diffuses and spreads over a large surface. It affords a “fresh effect” sensation under the fingers.

The applied makeup film reveals a “second skin” aspect; it is uniform and color-unifying. It has a matt appearance with “radiance” (returns light frozen or greasy appearance) and especially makes the skin look hydrated as if a cream had been applied. The skin relief is smoothed and the applied composition fills the pores without giving a pasty effect.

The applied film also has a soft feel without the feeling of dragging, and with no greasy or tacky effect.

### EXAMPLE 2

| Chemical name | weight % |
|---|---|
| Disodium ethylenediaminetetraacetate dihydrate | 0.1 |
| 4-(2-Hydroxyethyl)piperazin-1-ethanesulfonic acid (HEPES | |
| Buffer Grade from Applichem) | 1 |
| Citric acid monohydrate | 0.015 |
| Titanium oxide (untreated anatase) (CI: 77891) | 4.68 |
| Red iron oxide (CI: 77491) | 0.34 |
| Yellow iron oxide (CI: 77492) | 0.86 |
| Black iron oxide (CI: 77499) | 0.12 |
| 2-Phenoxyethanol | 0.5 |
| 2-Octyldodecanol | 0.5 |
| Xanthan gum | 0.1 |
| Polyacrylamidomethylpropanesulfonic acid (AMPS) partially | 0.2 |
| neutralized with aqueous ammonia and highly crosslinked | |
| (Hostacerin AMPS from Clariant) | |
| Polydimethylsiloxane (viscosity: 350 cSt) (Xiameter PMX- | 1 |
| 200 Silicone Fluid 350 CS from Dow Corning) | |
| Polydimethylsiloxane (viscosity: 100 cSt) (Xiameter PMX- | 1 |
| 200 Silicone Fluid 100 CS from Dow Corning) | |
| 96° denatured ethyl alcohol | 5 |
| Microbiologically clean deionized water | qs 100 |
| Glycerol | 7 |
| 1,2-Octanediol | 0.3 |
| Oxyethylenated (20 OE) methyl glucoside sesquistearate | |
| (Glucamate SSE-20 Emulsifier from Lubrizol) | 0.1 |

This composition is prepared in the same manner as for the composition of Example 1.

The product thus obtained has, on application to facial skin, a fluid, light, very slippery texture with a sensation of water that diffuses and spreads over a large surface. It affords a “fresh effect” sensation under the fingers.

The applied makeup film reveals a “second skin” aspect; it is uniform and color-unifying. It has a matt appearance with “radiance” (returns light frozen or greasy appearance) and especially makes the skin look hydrated as if a cream had been applied. The skin relief is smoothed and the applied composition fills the pores without giving a pasty effect.

The applied film also has a soft feel without the feeling of dragging, and with no greasy or tacky effect.

1. A cosmetic care and/or makeup composition comprising at least 65% by weight relative to its total weight of an aqueous phase, at least 2% by weight relative to its total weight of pigments in the form of pigmentary particles that are dispersed and/or dispersible in aqueous medium, at least one water-soluble or water-dispersible gelling agent in a content ranging from 0.05% to 40% by weight relative to the total weight of the composition, and at least one polyol containing from 3 to 12 carbon atoms, said composition having a viscosity at least equal to 600 Pa·s at a shear of 0.010 s⁻¹ and less than 0.8 Pa·s at a shear of 1000 s⁻¹, and said pigmentary particles being first formed from an intimate mixture of said pigments and from at least one polyol.

2. A cosmetic care and/or makeup composition comprising at least 65% by weight relative to its total weight of an aqueous phase, at least 2% by weight relative to its total weight of pigments in the form of pigmentary particles that are dispersed and/or dispersible in aqueous medium, at least one water-soluble or water-dispersible gelling agent in a content ranging from 0.05% to 40% by weight relative to the total weight of the composition, and at least one polyol containing from 3 to 12 carbon atoms, said composition having a viscosity at least equal to 600 Pa·s at a shear of 0.010 s⁻¹ and less than 0.8 Pa·s at a shear of 1000 s⁻¹, and
said gelling agent being chosen from modified or unmodified carboxyvinyl polymers, polyacrylates and polymethacrylates, optionally crosslinked and/or neutralized 2-acrylamido-2-methylpropanesulfonic acid polymers and copolymers, ethoxylated (25 EO) AMPS/stearyl methacrylate copolymers crosslinked with trimethylolpropane triacrylate (TMPTA), AMPS/acrylamide copolymers, polyacrylamides, associative polyurethanes, polysaccharides such as xanthan gum, xanthan derivatives, carrageenans, gellans, alginates, celluloses such as microcrystalline cellulose and cellulose polymers such as hydroxyethylcellulose, hydroxymethylcellulose, hydroxypropylcellulose, methylcellulose, ethylhydroxyethylcellulose and carboxymethylcellulose, and mixtures thereof.

3. The composition as claimed in claim 2, in which said particles are first formed from an intimate mixture of said pigments and from at least one polyol.

4. The composition as claimed in claim 1, in which said at least one water-soluble or water-dispersible gelling agent is present in a content ranging from 0.1% to 20% by weight 15% by weight relative to the total weight of the composition.

5. The composition as claimed in claim 1, in which said gelling agent is chosen from modified or unmodified carboxyvinyl polymers, polyacrylates and polymethacrylates, optionally crosslinked and/or neutralized 2-acrylamido-2-methylpropanesulfonic acid polymers and copolymers, ethoxylated (25 BO) AMPS/stearyl methacrylate copolymers crosslinked with trimethylolpropane triacrylate (TMPTA), AMPS/acrylamide copolymers, polyacrylamides, associative polyurethanes, polysaccharides selected from xanthan gum, xanthan derivatives, carrageenans, gellans, alginates, celluloses and cellulose polymers selected from hydroxyethylcellulose, hydroxymethylcellulose, hydroxypropylcellulose, methylcellulose, ethylhydroxyethylcellulose and carboxymethylcellulose, and mixtures thereof.

6. The composition as claimed in claim 1, in which said gelling agent is chosen from crosslinked and/or neutralized 2-acrylamido-2-methylpropanesulfonic acid polymers and copolymers or ethoxylated (25 EO) AMPS/stearyl methacrylate copolymers crosslinked with trimethylolpropane triacrylate (TMPTA).

7. The composition as claimed in claim 1, in which said pigments are dispersed in said composition in the form of pigmentary particles with a mean size of between 0.01 μm and 100 μm.

8. The composition as claimed in claim 1, comprising from 2% to 20% by weight of pigments relative to its total weight.

9. The composition as claimed in claim 1, in which the pigments are chosen from titanium, zirconium or cerium oxides or dioxides, zinc, iron or chromium oxides, ferric blue, manganese violet, ultramarine blue, chromium hydrate and untreated mixtures thereof.

10. The composition as claimed in claim 1, in which said particles are formed from a mixture of said pigments and from at least one polyol in a polyol(s)/pigments ratio ranging from 0.025 to 7.5.

11. The composition as claimed in claim 1, in which said particles are obtained after milling said mixture.

12. The composition as claimed in claim 1, in which said polyol is glycerol.

13. The composition as claimed in claim 1, comprising from 0.5% to 15% by weight, of polyol(s) relative to its total weight.

14. The composition as claimed in claim 1, in which the pigments are used in an aqueous phase/pigments weight ratio ranging from 3 to 45.

15. The composition as claimed in claim 1, also comprising at least one monoalcohol.

16. The composition as claimed in claim 1, comprising from 65% to 90% by weight of aqueous phase relative to its total weight.

17. The composition as claimed in claim 1, also comprising at least one fatty substance.

18. The composition as claimed in claim 1, characterized in that it is in the form of a foundation.

19. Method for making up and/or caring for a composition of keratin materials comprising at least one step of applying to said keratin material a composition as defined according to claim 1.

20. The composition as claimed in claim 2, in which said at least one water-soluble or water-dispersible gelling agent is present in a content ranging from 0.1% to 20% by weight relative to the total weight of the composition.

21. The composition as claimed in claim 4, in which said gelling agent is chosen from modified or unmodified carboxyvinyl polymers, polyacrylates and polymethacrylates, optionally crosslinked and/or neutralized 2-acrylamido-2-methylpropanesulfonic acid polymers and copolymers, ethoxylated (25 EO) AMPS/stearyl methacrylate copolymers crosslinked with trimethylolpropane triacrylate (TMPTA), AMPS/acrylamide copolymers, polyacrylamides, associative polyurethanes, polysaccharides selected from xanthan gum, xanthan derivatives, carrageenans, gellans, alginates, celluloses and cellulose polymers selected from hydroxyethylcellulose, hydroxymethylcellulose, hydroxypropylcellulose, methylcellulose, ethylhydroxyethylcellulose and carboxymethylcellulose, and mixtures thereof.

22. The composition as claimed in claim 2, in which said gelling agent is chosen from crosslinked and/or neutralized 2-acrylamido-2-methylpropanesulfonic acid polymers and copolymers or ethoxylated (25 EO) AMPS/stearyl methacrylate copolymers crosslinked with trimethylolpropane triacrylate (TMPTA).

23. The composition as claimed in claim 2, in which said pigments are dispersed in said composition in the form of pigmentary particles with a mean size of between 0.01 μm and 100 μm.

24. The composition as claimed in claim 2, comprising from 2% to 20% by weight of pigments relative to its total weight.

25. The composition as claimed in claim 2, in which the pigments are chosen from titanium, zirconium or cerium oxides or dioxides, zinc, iron or chromium oxides, ferric blue, manganese violet, ultramarine blue, chromium hydrate and untreated mixtures thereof.

26. The composition as claimed in claim 2, in which said particles are formed from a mixture of said pigments and from at least one polyol in a polyol(s)/pigments ratio ranging from 0.025 to 7.5.

27. The composition as claimed in claim 3, in which said particles are obtained after milling said mixture.

28. The composition as claimed in claim 2, in which said polyol is glycerol.

29. The composition as claimed in claim 2, comprising from 0.5% to 15% by weight of polyol(s) relative to its total weight.

30. The composition as claimed in claim 2, in which the pigments are used in an aqueous phase/pigments weight ratio ranging from 3 to 45.

31. The composition as claimed in claim 2, also comprising at least one monoalcohol.

32. The composition as claimed in claim 2, comprising from 65% to 90% by weight and in particular from 66% to 85% by weight of aqueous phase relative to its total weight.

33. The composition as claimed in claim 2, also comprising at least one fatty substance.

34. The composition as claimed in claim 2, characterized in that it is in the form of a foundation.

35. Method for making up and/or caring for a composition of keratin materials comprising at least one step of applying to said keratin material a composition as defined according to claim 2.
